(19) United States
(12) Patent Application Publication
(10) Pub. No.: US 2012/0197125 A1
(43) Pub. Date: Aug. 2, 2012

(54) ULTRASOUND DIAGNOSTIC DEVICE

(75) Inventors: Chihiro SHIBATA, Nasushiobara-shi (JP); Takuya SASAKI, Nasu-machi (JP); Kuramitsu NISHIHARA, Otawara-shi (JP); Akihiro KAKEE, Nasushiobara-shi (JP)

(73) Assignees: TOSHIBA MEDICAL SYSTEMS CORPORATION, OTAWARA-SHI (JP); KABUSHIKI KAISHA TOSHIBA, TOKYO (JP)

(21) Appl. No.: 13/357,878

(22) Filed: Jan. 25, 2012

(30) Foreign Application Priority Data
Jan. 27, 2011 (JP) 2011-014917
Dec. 22, 2011 (JP) 2011-280838

(51) Int. Cl. A61B 8/14

(52) U.S. Cl. 600/443

(57) ABSTRACT

As one illustrative embodiment, a first calculator obtains a first power value, which is the sum of the absolute value of a plurality of data obtained by a plurality of transmissions. A velocity-vector calculating unit obtains the velocity vector based on a plurality of data. A second calculator obtains a second power value, which is the absolute value of the velocity vector. A post-filter processor comprises one or more post-filter processes to determine the power value, and in the one or more post-filter processes, a power value other than the power value used in obtaining the threshold value for determination is selected as the power value at each observation point and the selected power value is determined based on the threshold value for determination.

### CROSS-REFERENCE TO RELATED APPLICATION

This application is based upon and claims the benefit of priority from Japanese Patent Application No. 2011-014917, filed Jan. 27, 2011 and No. 2011-280838, filed Dec. 22, 2011; the entire contents of all of which are incorporated herein by reference.

### FIELD

Embodiments of the present invention are related to an ultrasound diagnostic device and an image processing program thereof.

### BACKGROUND

Some conventional ultrasound diagnostic devices displays blood flow signals to be overlaid on a tomographic image (B mode image) in color as two-dimensional blood flow imaging (for example, Japanese Unexamined Patent Application Publication H4-218143).

The color display of this two-dimensional blood flow imaging allows color information to be related to the blood flow signals and allows two-dimensional blood flow imaging to be displayed by the color information, so that the blood flow may be visualized.

However, depending on the site, for example, insufficient sensitivity of a high frequency range occurs and blood flow signals for sufficient diagnosis are not obtained. In order to compensate for this lack of sensitivity, gain adjustment of the blood flow signal is possible; however, this also increases the blood flow signals and noise signals, resulting in the obtaining of only blood flow doppler signals buried in noise signals.

To visualize the blood flow by color information, an autocorrelation method is generally used to obtain the color information thereof. In order to form one raster of a tomographic image of the test object, it is necessary to transmit an ultrasonic wave a plurality of times in a same direction. A plurality of data may be obtained by a plurality of transmissions. A first power value, which is the sum of the absolute values of the obtained plurality of data, may be referred to as a scalar. A second power value, which is the absolute value of a velocity vector obtained from the obtained plurality of data, may be referred to as a vector, the ratio of both power values may be referred to as dispersion.

Conventionally, with a focus on their low power, noise signals select either the scalar or vector as the power value, carry out a blanking process that deletes power values not exceeding a predetermined threshold value, and use power values exceeding the threshold value as the color information for display processing that displays the blood flow.

However, when using the scalar, the sum of the absolute value of the signals is used without taking the effects of dispersion into consideration. Thus, when the blanking process is carried out, there was a problem of many noise signals remaining without being deleted, resulting in the display of a lot of noise.

Moreover, when using the vector, the effects of dispersion of the power value are taken into consideration, effectively deleting the noise signals. However, the power value of the blood flow declines at the same time. Thus, there was a problem of not being able to obtain a strong blood flow signal as when selecting the scalar.

The embodiment is intended to solve the abovementioned problems, with the objective of providing a diagnostic ultrasound diagnostic device that effectively eliminates unnecessary noise signals and obtaining a high-precision blood flow signal, as well as an image processing program thereof.

### BRIEF DESCRIPTION OF THE DRAWINGS

FIG. 1 is a block diagram indicating the ultrasound diagnostic device related to the first embodiment.

FIG. 2 is a diagram of a time change when ultrasonic waves are transmitted to the same place a plurality of times.

FIG. 3 is a diagram indicating the velocity vector represented on the complex plane.

FIG. 4 is a diagram indicating the scalar and vector sent from the log-compression unit to a post filter processor.

FIG. 5 is a block diagram indicating the doppler signal processing unit to be compared with the embodiment.

FIG. 6 is a block diagram indicating an example of the doppler signal processing unit.

FIG. 7 is a block diagram indicating an example of the calculator.

FIG. 8 is a block diagram indicating another example of the doppler signal processing unit.

FIG. 9 is a block diagram indicating another example of the doppler signal processing unit.

FIG. 10 is a diagram indicating a B mode image with an experimental phantom.

FIG. 11 is a blood flow image compared with the embodiment in which the scalar was post-filter processed by the threshold value of the scalar and simulated on the display by the scalar.

FIG. 12 is a blood flow image compared with the embodiment in which the vector was post-filter processed by the threshold value of the vector and simulated on the display by the vector.

FIG. 13 is a blood flow image related to the embodiment in which the vector was post-filter processed by the threshold value of the vector and simulated on the display by the scalar.

FIG. 14 is a validation diagram of the blood flow image.

FIG. 15 is a diagram indicating an example of the processes for selecting the scalar or vector.

FIG. 16 is a diagram indicating an example of post-filter processing consisting of a blanking process alone.

FIG. 17 is a diagram indicating an example of post-filter processing consisting of a smoothing process alone.

### DETAILED DESCRIPTION

### First Embodiment

Embodiments of the ultrasound diagnostic device are set forth with reference to each diagram.

The ultrasound diagnostic device 1 transmits ultrasonic waves to the test object and generates a doppler spectrum image showing the velocity of the moving target of the test object (blood flow) based on waves reflected from the test object.

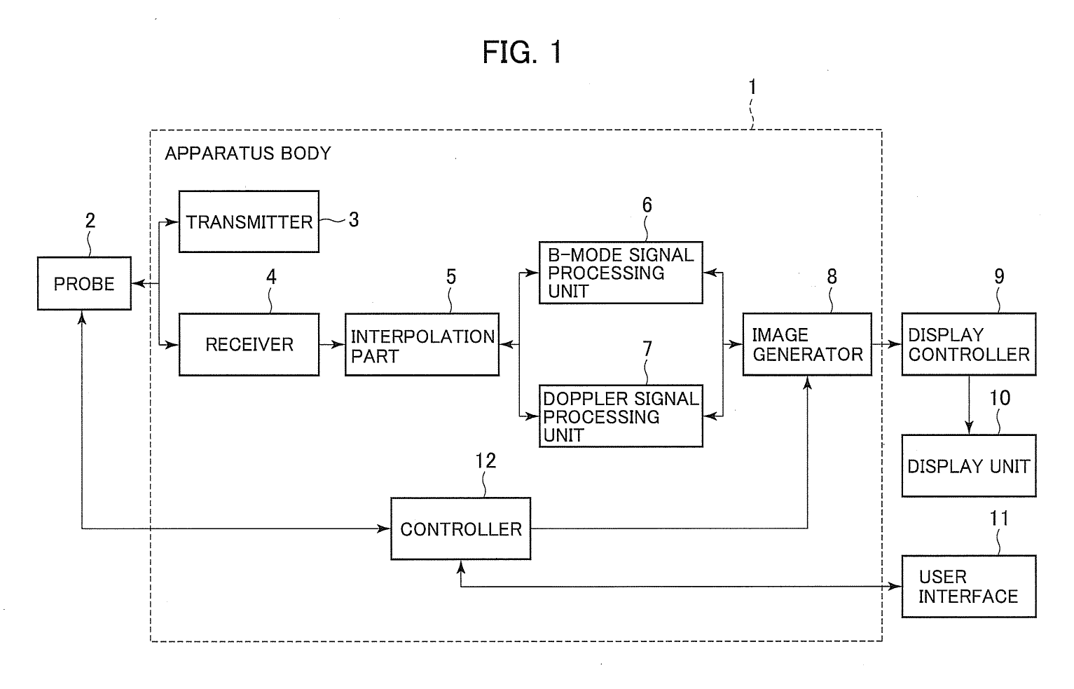

FIG. 1 is a block diagram indicating the ultrasound diagnostic device. As indicated in FIG. 1, the ultrasound diagnostic device 1 comprises an ultrasonic probe 2, a transmitter 3, a receiver 4, an interpolation part 5, a B-mode signal processing unit 6, a doppler signal processing unit 7, an image generator 8, a display controller 9, a display unit 10, a user interface 11, and a controller 12. The ultrasonic probe 2, the transmitter 3 and the receiver 4 are examples of the transceiver.

A one-dimensional array probe with a plurality of ultrasonic transducers arranged in a single row in a predetermined direction (scanning direction), or a two-dimensional array probe with the plurality of ultrasonic transducers arranged in a two-dimensional manner is used for the ultrasonic probe 2. Using the two-dimensional array probe, a three-dimensional region may be scanned by ultrasonic waves in order to obtain volume data in the three-dimensional region. Moreover, the one-dimensional array probe may be used for the ultrasonic probe 2, which is a one-dimensional array probe with the plurality of ultrasonic transducers arranged in a single row in the scanning direction allowing scanning of the three-dimensional region by mechanically oscillating the ultrasonic transducer in a direction parallel to the scanning direction.

The transmitter 3 generates ultrasonic waves by supplying electrical signals to the ultrasonic probe 2, while the receiver 4 receives the echo signals received by the ultrasonic probe 2. The transmitter 3 and the receiver 4 transmit and receive ultrasonic waves to and from the ultrasonic probe 2 according to a predetermined pulse repetition in frequency (PRF).

The transmitter 3 is provided with a clock generation circuit, a transmission delay circuit, and a pulsar circuit (not illustrated). The clock generation circuit is a circuit that generates a clock signal which determines the transmission timing and/or transmission frequencies of ultrasonic wave signals. The transmission delay circuit is a circuit that delays transmission of the ultrasonic waves and carries out transmission focus. The pulsar circuit has a pulsar corresponding to the number of individual channels corresponding to each ultrasonic transducer built in, generates a driving pulse at the delayed transmission timing, and supplies pulses to each ultrasonic transducer of the ultrasonic probe 2.

The receiver 4 is provided with a preamplifier circuit, an A/D conversion circuit, a reception delay circuit, and an adding circuit. The preamplifier circuit amplifies the echo signals emitted from each ultrasonic transducer of the ultrasonic probe 2 per receiving channel. The A/D conversion circuit A/D converts the amplified echo signals. The reception delay circuit provides a delay time necessary for determining the receiving directivity of the echo signals following the A/D conversion. The adding circuit adds the echo signal provided by the delay time. By the addition, a reflection component from the direction corresponding to the receiving directivity is emphasized. Moreover, at times, signals that have been adding-processed by the receiver 4 may be referred to as RF signals (Radiofrequency Signals).

The RF signals emitted from the receiver 4 are output to the interpolation part 5. The interpolation part 5 uses the RF signals emitted from the receiver 4 for interpolation by periodically estimating missing signals.

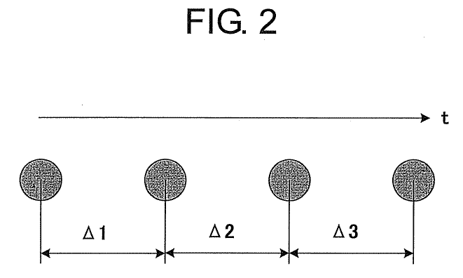

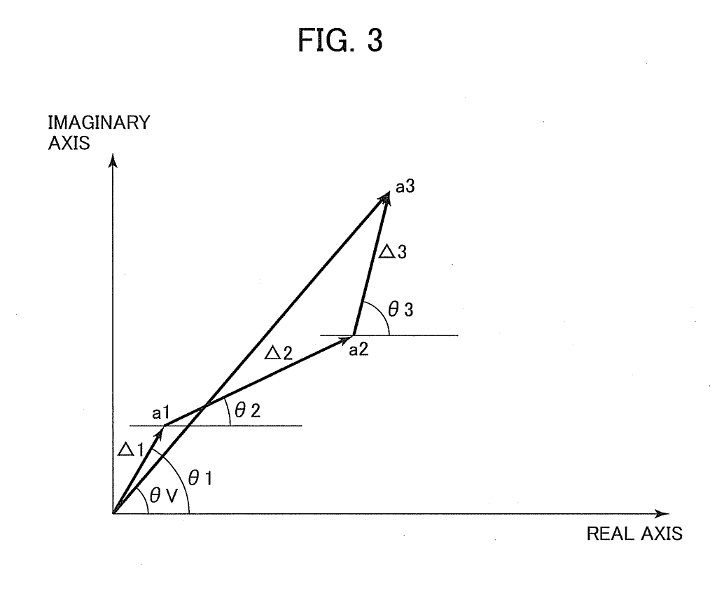

Next, the actions of the ultrasonic probe 2, the transmitter 3, the receiver 4, and the interpolation part 5 are briefly set forth with reference to FIG. 2 and FIG. 3.

The ultrasonic probe 2 is exposed to the body surface of the measurement site of the test object. The tester continuously scans the ultrasonic probe 2 along the body surface of the test object. During the scanning, transmission signals applied from the transmitter 3 are sent to each ultrasonic transducer (not illustrated) of the ultrasonic probe 2, and an ultrasonic pulse is transmitted from the ultrasonic probe 2 to the test object. The transmitted ultrasonic pulse is reflected from the test object as needed, enters the receiver 4 through the ultrasonic probe 2, so that amplification, A/D conversion, delay calculation, adding processing, etc., are carried out inside the receiver 4. Signals on which each process was carried out at the receiver 4 enter the interpolation part 5. The interpolation part 5 interpolates the missing signals based on the signals that entered.

FIG. 2 is a diagram indicating the change in time required in transmitting and receiving each time when ultrasonic wave are transmitted and received at the same position in the test object (observation point) four times. In FIG. 2, the horizontal axis is the time (t) axis, and the number of times the ultrasonic waves were transmitted and received from the first time to the fourth time is indicated by N1 to N4.

As indicated in FIG. 2, the changes in time required in transmitting and receiving the ultrasonic waves are subsequently Δ1, Δ2, and Δ3, indicating between the first and the second time, between the second and the third time, and between the third and the fourth time. The changes in required time Δ1, Δ2, and Δ3 are comparable with the velocity vector of the moving target at a series of observation points.

If the direction of the ultrasonic waves is a solid line, multiple observation points are provided on the solid line. The interpolation part 5 assumes an interpolation line adjacent to the solid line, arranges the observation points on the assumed interpolation line, and interpolates the change in required time regarding the arranged observation points.

For example, the interpolation part 5 interpolates the change in time required regarding each observation point on the interpolation line as the mean value of the change in time required regarding each observation point of the interpolation line or solid line at an equal distance from the interpolation line.

Next, the signals interpolated by the RF signals and the interpolation part 5 (including the change in required time) are converted to IQ data with completed quadrature detection, and are subsequently emitted to the B-mode signal processing unit 6 and the doppler signal processing unit 7.

The B-mode signal processing unit 6 visualizes the amplitude information of the echo and generates B-mode ultrasonic wave raster data from the echo signals. In concrete terms, the B-mode signal processing unit 6 carries out filter processing of the quadraturely-detected data, and furthermore, carries out compression processing by logarithmic transformation.

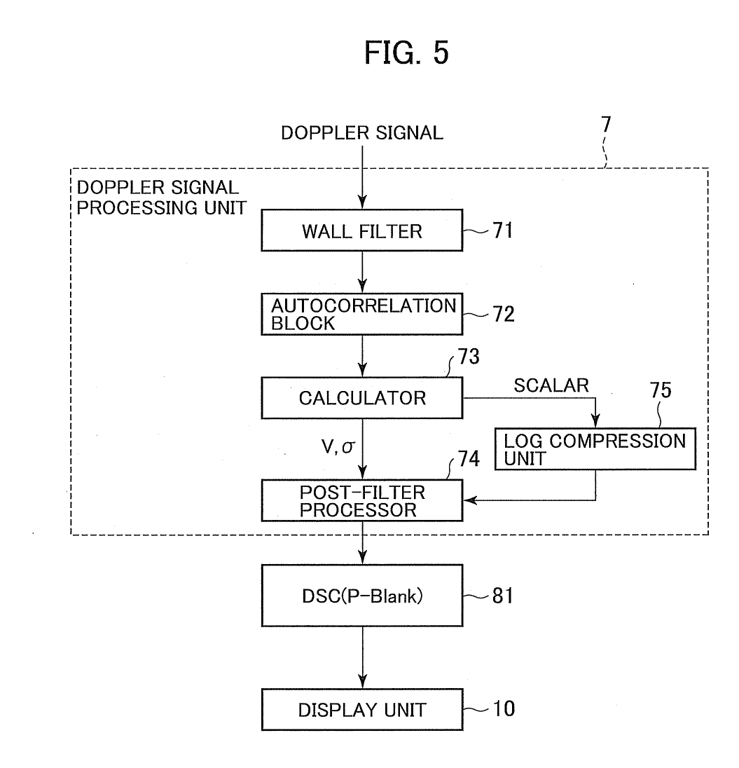

Next, the doppler signal processing unit 7 is explained with reference to FIG. 5.

FIG. 5 is a block diagram of the doppler signal processing unit 7 to be compared with the present embodiment. As indicated in FIG. 5, the doppler signal processing unit 7 is provided with a wall filter 71, an autocorrelation block (AC: Auto Correlator) 72, a calculator 73, a post-filter processor 74, and a log compression unit 75. The doppler signals (real-part component and imaginary-part component) that are carried in have clutter components removed by a wall filter 71, pass through an autocorrelation block 72, and are used to calculate the velocity V, dispersion σ, and power value P at the calculator 73. The power value P is compressed into an eight-bit signal by a log-compression unit 75. Moreover, before and after compression by the log-compression unit 75 are also explained by assigning P, P1, and P2 to the power value, scalar, and vector, respectively.

Here, post-filter processing refers to the filter process after the velocity V, dispersion σ, and power value P have been computed. The post-filter processor 74 comprises a blanking processor 741 and/or a smoothing processor 742. Moreover, at times, post-filter processing may refer to the blanking process and/or the smoothing process.

When post-filter processing comprises the blanking process or the smoothing process, either means may come first regarding the processes thereof.

Moreover, the post-filter processor 74 of this embodiment is explained as comprising the blanking processor 741 and not the smoothing processor 742.

The velocity V, dispersion σ, and power value P following compression are sent to a digital scan converter 81 (hereinafter, referred as DSC) via the blanking processor 741. Subsequently, in the DSC 81, they are converted to information displayed on the display unit 10, sent to the display unit 10, and displayed as color information. Details regarding the calculator 73, the blanking processor 741, the velocity V, the dispersion σ, and the power value P are mentioned later.

However, the doppler signal processing unit 7 indicated in the above FIG. 5 leads the power value P of either one of the scalar P1 or the vector P2 from the calculator 73 to the latter part. In contrast, the doppler signal processing unit 7 related to the present embodiment leads the power value P of both the scalar and the vector from the calculator 73 to the latter part, and selectively uses the power value of either the scalar or the vector in all processes using the power value of the latter part.

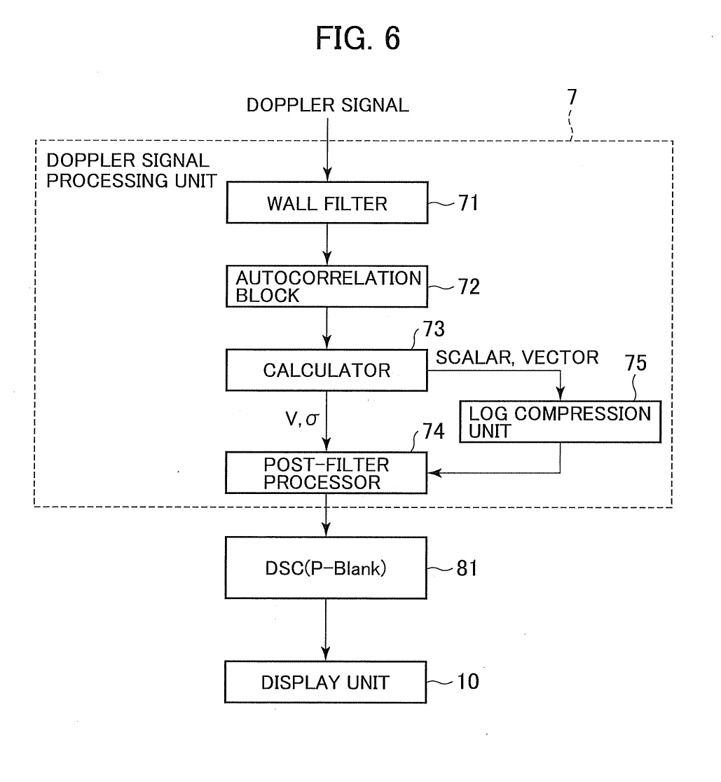

Next, the doppler signal processing unit 7 related to the present embodiment is explained with reference to FIG. 3 and FIG. 6.

The autocorrelation block 72 of the doppler signal processing unit 7 is an example of a velocity-vector calculating unit that obtains the velocity vector of the moving target (blood flow) based on the doppler signal obtained by the plurality of transmissions and receptions at each observation point in the test object. The doppler signal may be referred to as complex data (containing a real-part component and an imaginary-part component).

FIG. 3 is a diagram showing the changes in time required in the plurality of transmissions and receptions on a complex plane. The Δ1, Δ2, and Δ3 shown in FIG. 3 indicate the changes in time required for the plurality of transmissions and receptions, wherein |Δ1|, |Δ2|, and |Δ3| indicate the signal level size, and θ1, θ2, and θ3 indicate velocity components.

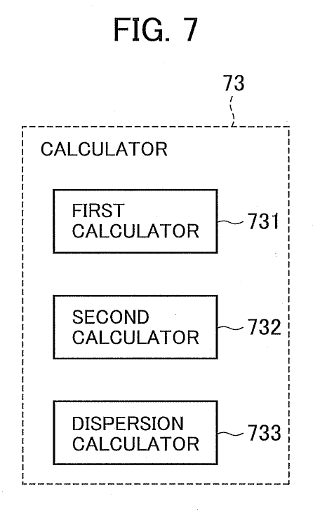

FIG. 6 is a diagram indicating one example of the doppler signal processing unit, wherein the scalar P1 and vector P2 flow from the calculator 73 to the latter part, and FIG. 7 is a block diagram showing an example of the calculator. As indicated in FIG. 6 and FIG. 7, the calculator 73 comprises the first calculator 731 that obtains the scalar P1, which is the sum of the absolute value of the plurality of complex data obtained from the plurality of transmissions and receptions, the second calculator 732 that obtains the vector P2, which is the absolute value of the velocity vector obtained from the autocorrelation block 72, and a dispersion calculator 733 that obtains the dispersion σ, which is the proportion of the scalar P1 and vector P2.

The first calculator 731 obtains the scalar P1 as the sum of the absolute value of complex data obtained by the plurality of transmissions and receptions.

The first calculator 731 outputs the obtained scalar P1 to the log-compression unit 75. The log-compression unit 75 compresses the scalar P1 and outputs it to the blanking processor 741.

Next, when the vector P2 obtained by the second calculator 732 is expressed by a numerical formula, it becomes the following formula (1):

P=|Δ1+Δ2+Δ3| (1)

The second calculator 732 outputs the obtained vector P2 to the log-compression unit 75. The log-compression unit 75 compresses the vector P2 and outputs it to the blanking processor 741.

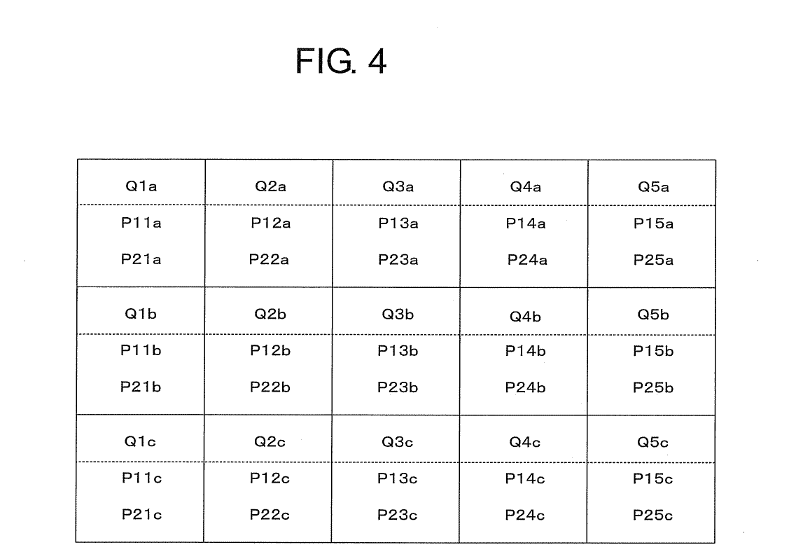

FIG. 4 is a diagram indicating the scalar P1 and vector P2 output from the log-compression unit 75 to the blanking processor 741. In FIG. 4, the observation points of the depth direction are indicated by (Q1a, Q1b, Q1c), (Q2a, Q2b, Q2c), etc. and the observation points of the raster direction are indicated by (Q1a, Q2a, Q3a, Q4a, Q5a), (Q1b, Q2b, Q3b, Q4b, Q5b), etc. Moreover, the scalar P1 of each observation point is indicated by P11a to P15c, while the vector P2 of each observation point is indicated by P21a to P25c.

The threshold values obtained from the scalar P1 and vector P2 are stored by a memory unit (not illustrated). Each threshold value is based on a rule of thumb. In response to the operation of an input unit of a user interface 11, the controller 12 causes the memory unit to store the threshold value. It should be noted that the controller 12 may obtain the threshold value using the predetermined formula from the input of the scalar P1 and vector P2, so that the threshold may be stored by the memory unit.

The calculator 73 has a dispersion calculator 733 to obtain the dispersion σ. The dispersion σ is the ratio determined based on the scalar P1 and vector P2, and is expressed by the following formula:

σ={P2/(Amount of data when making P2)}/{P1/(Amount of data when making P1) (2)

The doppler signal processing unit 7 comprises the blanking processor 741 that blanking-processes the power value at each observation point. One or a plurality of blanking processes is included in the blanking processor 741. In each blanking process, the scalar P1 or vector P2 is used as the blanking-processed power value. The scalar P1 or the vector P2 is used as the threshold value when determining the numerical power value (determining the threshold value). In the present embodiment, when either of the scalar P1 or vector P2 is selected as the blanking-processed power value in at least one or more blanking processes, the other of the scalar P1 or vector P2 is used as the threshold value towards the selected power value, and power values less than the threshold value are deleted. It should be noted that regarding other blanking processes, the blanking-processed power value and the power value used to determine the threshold value may be the same scalar P1 or the same vector P2.

Next, an example of the post-filter process using the scalar P1 as the blanking-processed power value, and using the vector P2 to determine the threshold value of the blanking-processed power value is shown.

When the scalar P11a and P11b of the observation points Q1a and Q1b are under the threshold value (luminance value: 100) and when the scalars P11c to P15c of other observation points exceed the threshold value, the blanking processor 741 deletes the scalars P11a and P11b from the memory unit (such value is determined as 0), while the other scalars P11c to P15c remain in the memory unit.

Furthermore, the embodiment above is indicated such that it uses the vector P2 as the threshold value and blanking-processes the scalar P1, but an opposite mode is possible; in other words, the scalar P1 may be used as the threshold value and the vector P2 may be blanking-processed.

### ALTERNATIVE EXAMPLE 1

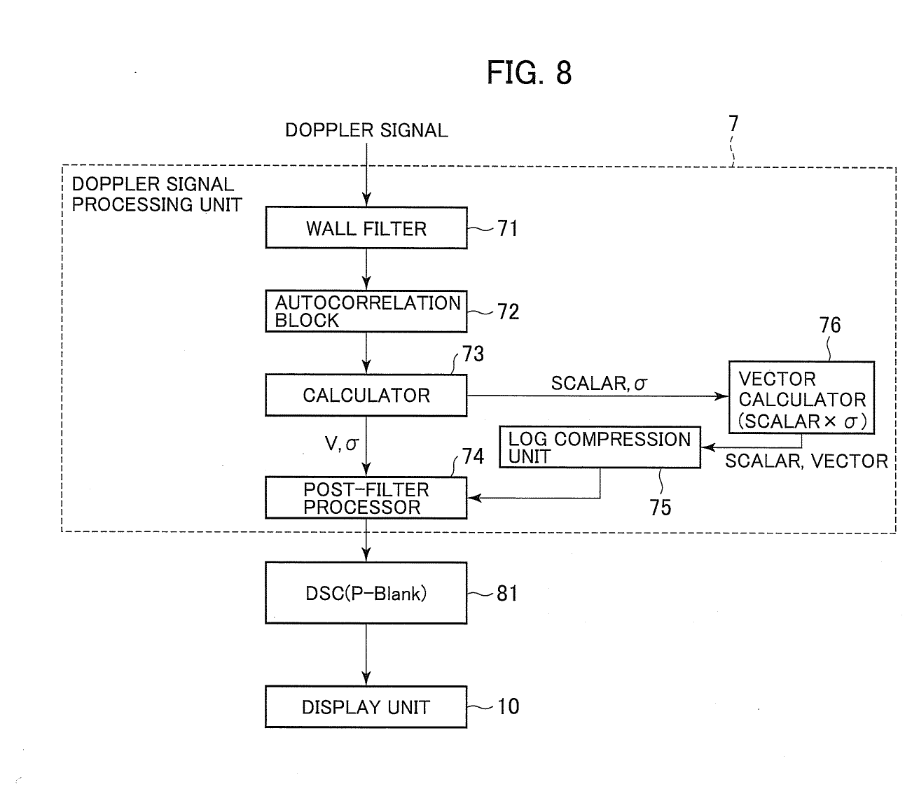

Next, other examples of the doppler signal processing unit are explained with reference to FIG. 8. It should be noted that the post-filter processor 74 of the alternative examples are set forth as including the blanking processor 741 and not the smoothing processor 742.

FIG. 8 is a block diagram indicating other examples of the doppler signal processing unit. As indicated in FIG. 8, the doppler signal processing unit 7 has a vector calculator 76. The scalar P1 and dispersion σ are input from the calculator 73 to the vector calculator 76, while the vector calculator 76 calculates the vector P2 based on the scalar P1 and dispersion σ.

The vector P2 is obtained by the following formula:

P2=P1

Here, σ is obtained using the above formula (2).

The scalar P1 and the obtained vector P2 are output into the log-compression unit 75 and subsequently blanking-processed by the blanking processor 741 in the same manner as the above embodiment, so explanations thereof are omitted. It should be noted that the dispersion c multiplied by the scalar P1 does not need to be itself. For example, those converted to an S-shape, convexed upwards, or convexed downwards may be used.

### ALTERNATIVE EXAMPLE 2

In the above Alternative Example 1, the dispersion σ was stored in the memory unit, but the dispersion σ may not be stored due to constraints on the capacity of the memory unit.

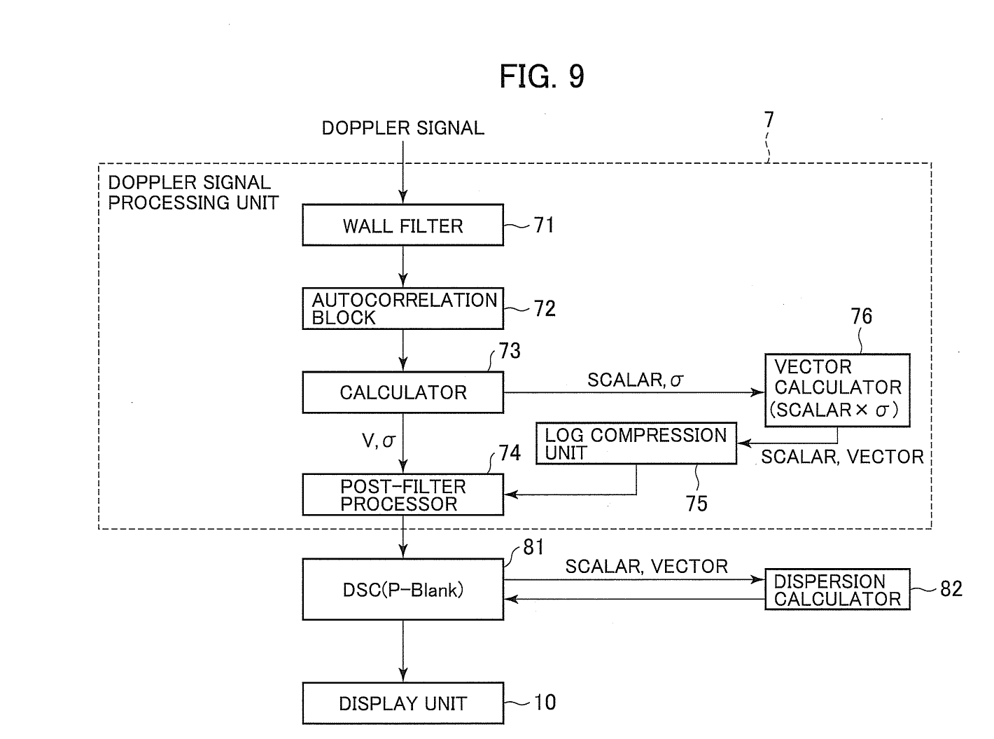

Next, an example of obtaining the dispersion σ by arithmetic is explained with reference to FIG. 9.

FIG. 9 is a block diagram indicating an example of a method of obtaining the dispersion σ based on the scalar P1 and vector P2. As shown in FIG. 9, the scalar P1 and vector P2 sent to the DSC 81 are sent to the dispersion calculator 82.

The dispersion calculator 82 carries out the following formula (3) by division following anti log processing of the scalar P1 and vector P2:

σ=A Log(vector)/A Log(scalar) (3)

Here, ALog refers to anti-log processing.

The abovementioned arithmetic-processed dispersion σ is sent to the DSC 81 and the scalar P1 and vector P2 are converted based on the dispersion σ.

Signals emitted from the B-mode signal processing unit 6 and the doppler signal processing unit 7 explained above are sent to the image generator 8.

The image generator 8 generates ultrasonic wave image data based on data that has been processed at the B-mode signal processing unit 6. For example, the image generator 8 comprises the DSC81, and converts the data that has been processed at the B-mode signal processing unit 6 to an image data that is expressed by a rectangular coordinate system in order to obtain the image expressed by the rectangular coordinate system. For example, the image generator 8 generates tomographic data as two-dimensional information based on B-mode ultrasonic wave raster data, outputting the tomographic data to the display controller 9. The display controller 9 causes the display unit 10 to display the tomographical image based on the tomographic data.

The image generator 8 generates two-dimensional blood flow imaging based on the data that has been processed at the doppler signal processing unit 7. The image generator 8 color-processes the data sent from the doppler signal processing unit 7. Moreover, the data sent from the doppler signal processing unit 7 comprises the scalar P1 of each observation point left behind at the blanking process and the scalar P1 of the observation points deleted during the blanking process (corresponding luminance value: 0).

During the color process, for example, when the relation of average velocity—dispersion (V-σ) is displayed, the blood flow approaching the ultrasonic probe 2 is converted to a red-based color, and the blood flow receding from the ultrasonic probe 2 is converted to a blue-based color. Moreover, the magnitude of the average velocity V is expressed by the difference in luminance. Furthermore, the dispersion a is expressed as a hue. The two-dimensional blood flow image is output to the display controller 9. The display controller 9 causes the display unit 10 to display the two-dimensional blood flow image to be overlapped on the tomographical image.

When the controller 12 receives the coordinate information of each observation point (range gate) assigned by the operator from the user interface 11, the coordinate information of each observation point is output to the probe 2 or the image generator 8.

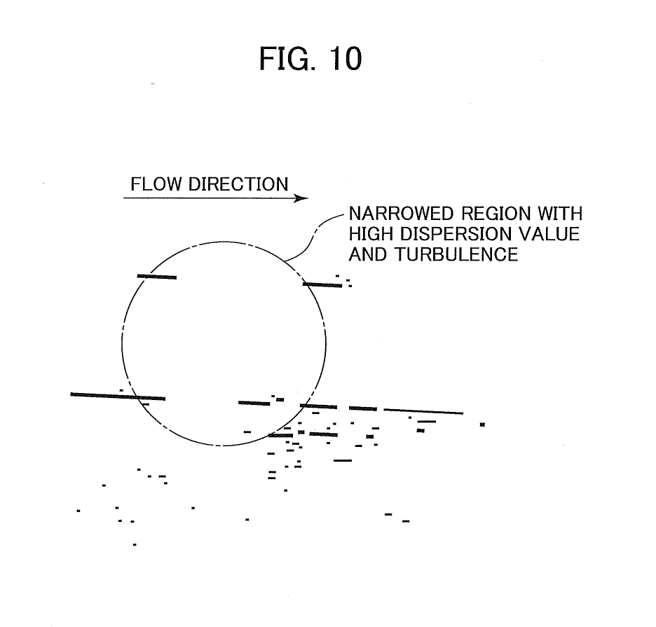

Next, the blood flow image indicated by the simulation is set forth with reference to FIG. 10 to FIG. 13.

FIG. 10 is a diagram indicating the B-mode image using an experimental phantom. As indicated in FIG. 10, the part enclosed within a circle is narrowed, meaning that blocking smooth flow of the blood flow causes turbulence and creates a region with a high dispersion value.

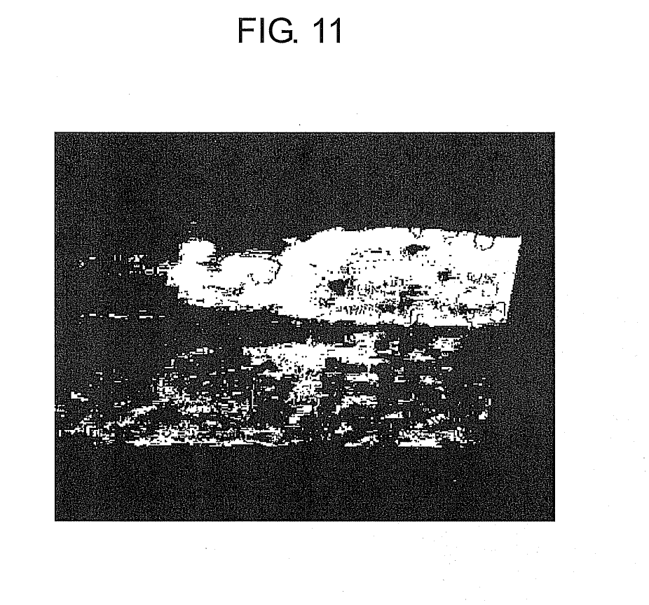

FIG. 11 is a blood flow image with simulation by using a scalar P1 for the threshold value, blanking-processing the scalar P1 (scalar blank), and displaying with the scalar P1 (scalar display). As indicated in FIG. 11, a great deal of noise is observed at the bottom end of the ROI (Region of Interest). In this manner, at the scalar blank and the scalar display, a great deal of noise is produced in exchange for displaying the blood flow, which is difficult to delete.

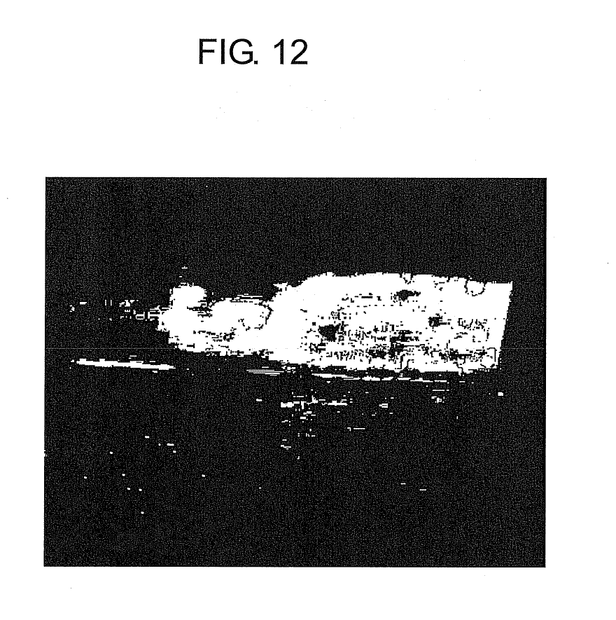

FIG. 12 is a blood flow image with simulation by using the vector P2 for the threshold value, blanking-processing the vector P2 (vector blank), and displaying with the vector P2 (vector display). As indicated in FIG. 12, the noise at the bottom end of the ROI is reduced, leaving only the blood flow components. However, the fundamental blood flow component is also weak compared to the scalar display (dark on a color map), and it may be observed that ideal blood flow information is not obtained.

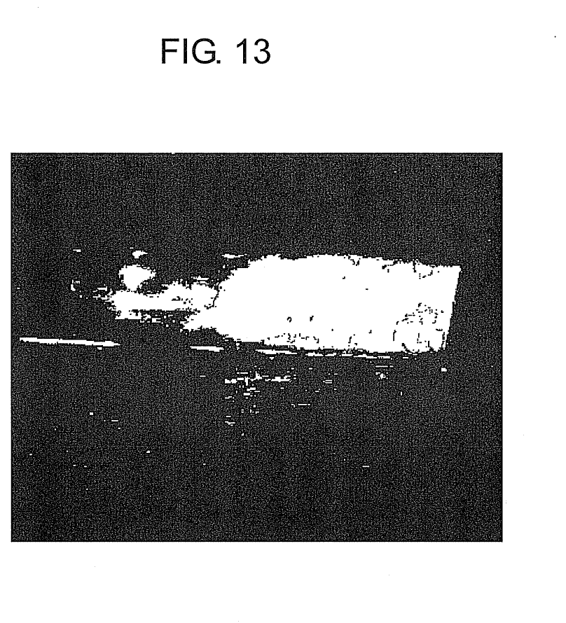

FIG. 13 is the present embodiment and is a blood flow image simulation by using a vector P2 for the threshold value, blanking-processing the scalar P1 (scalar blank), and displaying with the scalar P1 (scalar display). As indicated in FIG. 13, there is little noise at the bottom end of the ROI, and moreover, the noise as the power value of the blood flow component that is fundamentally desired becomes higher (bright on the color map) and it may be observed that ideal blood flow information is obtained.

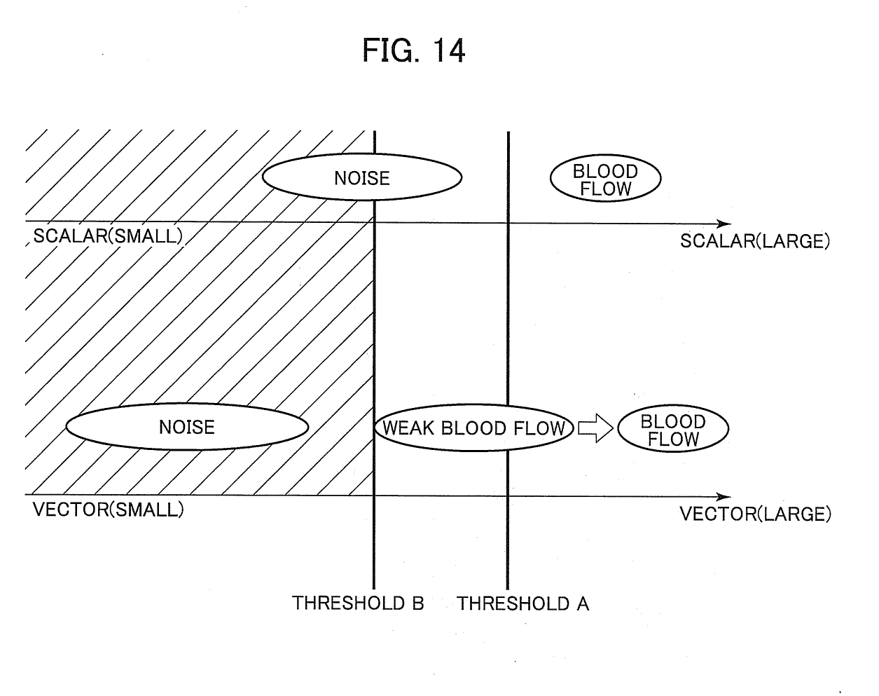

Next, a comparison between the scalar blank—the scalar display effect indicated in FIG. 11 and the scalar blank—the scalar display effect indicated in FIG. 13 is set forth with reference to FIG. 14. FIG. 14 is a validation diagram of the blood flow image. As indicated in FIG. 14, the scalar blank, which blanking-processes the scalar P1 by a threshold A of the scalar, deletes weak blood flows when deleting noise.

In contrast, the scalar blank, which blanking-processes the scalar P1 with a threshold value B of the vector, cannot completely delete the noise but can leave behind the weak blood flows. The ideal blood flow information may be obtained by scalar-displaying the blood flow containing this weak blood flows.

### Second Embodiment

In the second embodiment, the same number is attached to those items with the same composition as the first embodiment, so explanations thereof are omitted.

The first embodiment indicates that the post-filter processor 74 comprises a blanking processor 741 and does not comprise a smoothing processor 742. In contrast, the post-filter processor 74 of the second embodiment will be set forth as comprising both a blanking processor 741 and a smoothing processor 742. It should be noted that the post-filter process referred to in the following explanation is assumed to comprise both the blanking process and the smoothing process. Furthermore, as also mentioned in the first embodiment, when post-filter processing comprises the blanking process and the smoothing process, any of the processes thereof may come first.

The abovementioned post-filter processor 74 may properly use the scalar P1 and the vector P2 in calculations using the power value.

Moreover, the post-filter processor 74 may comprise a switchover part (not illustrated) for switching over between the power values (scalar, vector) used as the threshold value and the power values (scalar, vector) that are post-filter processed.

Next, the diagnosis subject of the test object is used as an example of the conditions. The switchover part switches over the power value used as the threshold value from one of the scalar P1 and the vector P2 to another in correspondence with the diagnosis subject of the test object. Moreover, the switchover part switches over the power value that is post-filter processed from one of the scalar P1 and the vector P2 to another.

Next, as another example of the conditions, dispersion σ may be used to switch over the power values in correspondence with the dispersion σ. For example, the display controller 9 causes the display unit 10 to display the dispersion σ, receives the entered dispersion σ, wherein the switchover part changes over from the scalar P1 to the vector P2 if the dispersion σ was large, and changes over from the vector P2 to the scalar P1 if the dispersion σ was small.

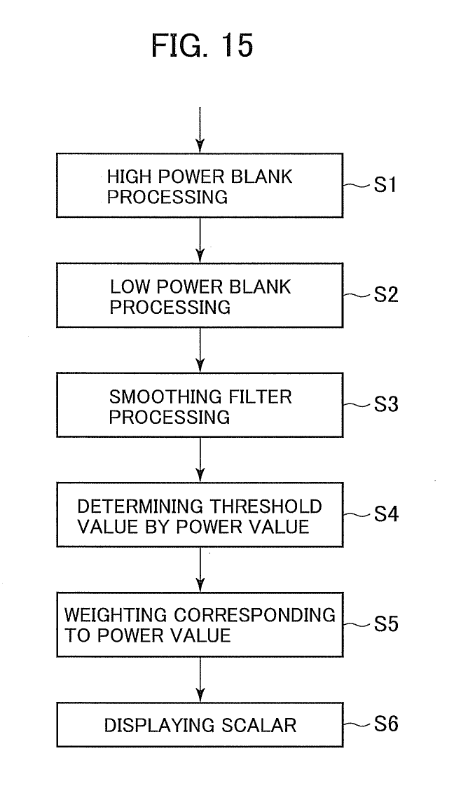

Next, an example of the process of selecting the scalar P1, vector P2 by the post-filter processor 74 is explained with reference to FIG. 15.

FIG. 15 is a diagram indicating an example of the processes for selecting the scalar P1 and vector P2. As indicated in steps S1 to S3 in FIG. 15, the post-filter processor 74 has a processing block of a high power blank and a low power blank as the blanking processor 741 and a processing block of a smoothing filter as the smoothing processor 742.

In the present embodiment, when either of the scalar P1 or the vector P2 is selected for the three power values to be post-filter processed, another of the scalar P1 or the vector P2 is used to determine the threshold regarding at least one among the three power values to be post-filter processed.

For example, when scalar P1 is selected for the three power values to be post-filter processed, the vector P2 is used for determining the threshold value of the high power blank among the three post-filter processes, and the scalar P1 is used for determining the threshold value of the low power blank and the smoothing filter, which are the other post-filter processes.

Moreover, for example, when vector P2 is selected for the three power values to be post-filter processed, the scalar P1 is used for determining the threshold value of the high power blank and the low power blank among the three post-filter processes, and the vector P2 is used for determining the threshold value of the smoothing filter, which is another post-filter process.

Moreover, the velocity V and dispersion a information may be revised by the power value, so vector P2 may also be used for the threshold value of the filter regarding the velocity V and the dispersion σ.

As indicated in steps S4 to S5 in FIG. 15, the DSC 81 also carries out coordinate transformation of the velocity V and the dispersion σ. Regarding the velocity component, the threshold value may be determined by the power value in the DSC 81 as well (S4), and weighting corresponding to the power value may be carried out and revised (S5).

Either of the scalar P1 or the vector P2 may be used for the weighted threshold value. The converted velocity data and dispersion data is sent to the display unit 10 in the same manner as the scalar P1 and the vector P2.

The DSC 81 sends the displayed data to the display unit 10. When power-displaying, the display unit 10 displays the scalar P1 (step S6). By means of steps S1 to S6 mentioned above, components with a high dispersion value and a high suspicion of noise may effectively removed so that the ideal blood flow display may be obtained regarding areas of normal flow.

Furthermore, in the embodiment, as indicated in FIG. 15, the blanking process (S1, S2) is carried out in advance regarding the three post-filter processes, and the smoothing process (S3) is subsequently carried out. However, as mentioned above, any of the processes thereof may come first. For example, the smoothing process may be carried out first and the blanking process may be carried out following that.

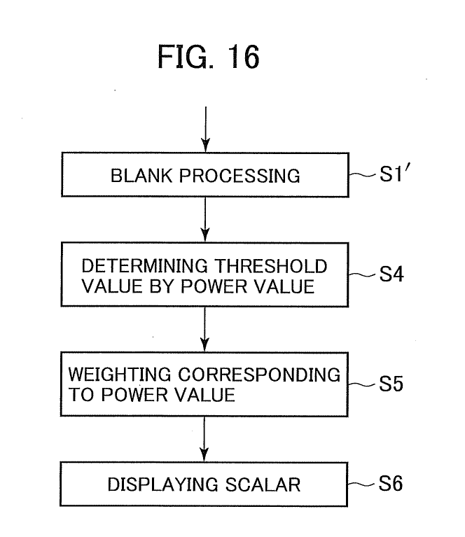

FIG. 16 is a diagram indicating post-filter processing consisting of the blanking process alone. As indicated in FIG. 16, the blanking process (S1′) may be carried out as post-filter processing without carrying out the smoothing process and may be transferred to step S4 that determines the threshold value by a power value.

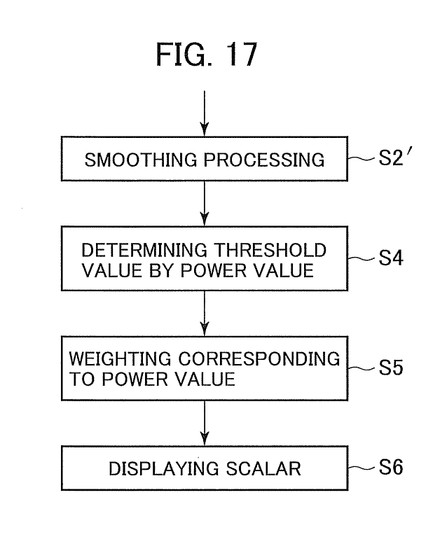

FIG. 17 is a diagram indicating an example of post-filter processing consisting of the smoothing process alone. As indicated in FIG. 17, the smoothing process (S2′) may be carried out as post-filter processing without carrying out the blanking process and may be transferred to step S4 that determines the threshold value by a power value.

Moreover, in the embodiment, the scalar P1 and/or the vector P2 were indicated as the power values that are post-filter processed. But without limiting this, the autocorrelation coefficient may also be post-filter processed. The embodiment set forth usage of the power value to be post-filter processed, while each embodiment defines an observation value as a generic term including the power value and the autocorrelation coefficient.

The autocorrelation coefficient is expressed by the following formula:

Δk=ak+ibk(k=1, 2, . . . , n)

Δk=ak/|ak|+i bk/|bk|

Here, n corresponds to the amount of data obtained from the plurality of sending and receiving. The autocorrelation coefficient (complex data after Δk is determined from IQ data) is suitable to be retained as data since the volume of the data is little. To post-filter the complex data is advantageous in more accurate than filter-processing the absolute value data.

The threshold value regarding the scalar P1 or vector P2 indicated in the embodiment may be entered by the operator using the user interface 11 based on the scalar P1, vector P2, and dispersion σ displayed on the display unit 10. Moreover, the threshold value obtained from a formula predetermined based on the scalar P1 or vector P2 may be automatically entered.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of embodiments described herein may be made without departing from the spirit of the inventions. The accompanying claims and their equivalents are intended to cover such forms or modifications as would fall within the scope and spirit of the inventions.

What is claimed is:

1. An ultrasound diagnostic device, comprising:
a transceiver configured to transmit and receive ultrasonic waves to a plurality of observation points in a test object by multiple times,
a first calculator configured to obtain a first power value, which is the sum of the absolute values of a plurality of data obtained by the plurality of transmissions and receptions,
a velocity-vector calculator configured to obtain the velocity vector of a moving target at each observation point based on the obtained plurality of data,
a second calculator configured to obtain a second power value, which is the absolute value of the obtained velocity vector,
a memory unit configured to store in advance a threshold value for determination based on at least one of the first power value and the second power value at each observation point.
a post-filter processor configured to determine an observation value among observation values except the power value at the observation points used for the threshold value;
an image generator configured to generate an image of the moving target based on the post-filter processed observation values at each observation points; and
a display controller configured to cause a display unit to display the created image of the moving target.

2. The ultrasound diagnostic device according to claim 1, further comprising a plurality of the post-filter processors; wherein one of the first power value and the second power value is used as the threshold value for determination for each post-filter processor.

3. The ultrasound diagnostic device according to claim 2, wherein the plurality of the post-filter processors comprise a blanking process and a smoothing process,
the threshold value for determination is a first threshold based on the first power value and the second threshold based on the second power value,
the blanking process uses one of the first threshold value and the second threshold value and deletes the selected power values that are less than the threshold value, and
the smoothing process uses one of the first threshold value and the second threshold value to process the selected power value based on the threshold value.

4. The ultrasound diagnostic device according to claim 2, wherein the post-filter processor uses the first power value or the second power value corresponding to the diagnosis subject of the test object as the power value and the threshold value at the each observation point.

5. The ultrasound diagnostic device according to claim 2, wherein the display controller causes the display unit to visibly display the dispersion, which is the ratio of the first power value and the second power value, and
the post-filter process uses the first power value or the second power value corresponding to the dispersion as the power value and the threshold value in the each observation point.

6. A computer program product embodied on a non-transitory computer readable medium, comprising:
computer code for obtaining a first power value, which is the sum of the absolute values of a plurality of data obtained by the plurality of transmissions and receptions when transmitting and receiving ultrasonic waves to a plurality of observation points in the test object by a plurality of times,
computer code for obtaining the velocity vector of a moving target at each observation point based on the obtained plurality of data,
computer code for obtaining a second power value, which is the absolute value of the obtained the velocity vector,
computer code for storing in advance a threshold value for determination obtained by using at least one of the first power value and the second power value at each observation point,
computer code for a post-filter processing comprising selecting a power value other than the power value used for the threshold value for determination as the power value at each observation point, and determining the selected power value based on the obtained threshold value for determination,
computer code for generating an image of the moving target based on the post-filter processed observation values at the observation points; and
computer code for displaying the created image of the moving target.
